(19) United States
(12) Patent Application Publication
(10) Pub. No.: US 2026/0272992 A1
(43) Pub. Date: Sep. 17, 2026

(54) Camelid-Derived Bioactive Compounds: Compositions, Methods, and Therapeutic Analgesic Applications

(71) Applicant: Applied Bio Research Inc., Windsor (CA)

(72) Inventors: Sabah A. A. Jassim, Windsor (CA); Khaldoun Rasem Shnewer, Amman (JO); Maryam Jassim, Windsor (CA); Hassan El-Cheikh, Windsor (CA); Richard M. Carlton, New York, NY (US)

(21) Appl. No.: 19/566,390

(22) Filed: Mar. 13, 2026

Related U.S. Application Data
(60) Provisional application No. 63/771,523, filed on Mar. 13, 2025.

(51) Int. Cl. A61K 35/35; A61K 9/00; A61K 31/16; A61K 31/232; A61P 25/02; C07C 231/24; C07C 233/18

(52) U.S. Cl. CPC A61K 35/35; A61K 9/0014; A61K 31/16; A61K 31/232; A61P 25/02; C07C 231/24; C07C 233/18

(57) ABSTRACT

A novel method for preparing and utilizing camel fat as a source of endocannabinoid compounds is described. This method involves isolating, quantifying, and formulating bioactive lipids, including palmitoylethanolamide (PEA), N-arachidonoylethanolamine (AEA), 2-arachidonoylglycerol (2-AG), and oleoylethanolamide (OEA), into a topical analgesic. A further method includes using the camelid-derived bioactive lipids for non-opioid pain management. The application of camelid-derived endocannabinoids contributes to the development of bioactive lipid therapeutics, providing a natural alternative to synthetic and plant-based analgesics.

### CROSS-REFERENCE TO RELATED APPLICATIONS

This application claims priority to U.S. Provisional Patent Application No. 63/771,523 entitled “Camelid-Derived Bioactive Compounds: Compositions, Methods, and Therapeutic Analgesic Applications,” filed Mar. 13, 2025. The entirety of that provisional application is incorporated herein by reference.

### FIELD OF THE INVENTION

The present invention relates to analgesics, and more specifically to endocannabinoids from camel fat.

### BACKGROUND OF THE INVENTION

Palmitoylethanolamide (PEA) is a naturally occurring N-(long-chain-acyl)ethanolamine and an ethanolamide derivative of palmitic acid (hexadecanoic acid). As a bioactive lipid, PEA plays diverse pharmacological roles, including functioning as an anti-inflammatory agent, antihypertensive compound, neuroprotective substance, and anticonvulsant. As a member of the endocannabinoid family and a functional analog of hexadecanoic acid, PEA has garnered significant scientific interest due to its wide-ranging therapeutic potential. Studies have highlighted its ability to mitigate stress responses and protect against anxiety and stress-induced neurochemical changes (Locci and Pinna, 2019).

Although analgesics are traditionally derived from plant-based compounds (Lisa et al., 2020; Agour et al., 2023; Amerikanou and Papada, 2023), bioactive substances from animal sources have demonstrated remarkable potency and specificity. Examples include analgesic peptides from leeches (Wang et al., 2023), cone snail venoms (Ramiro et al., 2022), and toxins from spiders and scorpions (Diochot 2021). However, animal fats, a relatively underexplored source, also exhibit potential analgesic properties. Omega-3 fatty acids from fish oil are well-documented for their pain-relieving effects (Zainal et al., 2009), while lard, historically used in traditional remedies, has demonstrated anti-inflammatory activity attributed to bioactive compounds such as 5-dodecanolide (Capó et al., 2021). These findings suggest that animal fats could play a role in modulating inflammatory pathways and pain perception.

### Biochemical and Structural Composition of Camelid Adipose Deposits

Unlike the subcutaneous and intramuscular fat storage mechanisms observed in humans and most other mammals, the adipose reserves in Camelus species are predominantly localized within the dorsal hump. This specialized fat storage adaptation enables camels to sustain prolonged periods of resource scarcity, with the adipose mass in a well-nourished adult individual reaching up to 35 kilograms. Lipid deposits are also present in various biological matrices, including the milk, blood, muscle tissue, and osseous structures of the camel. Notably, the extraction of hump-derived adipose tissue can be achieved through liposuction, permitting the preservation of the animal and the continued synthesis of lipid reserves.

The structural composition of camel hump fat is distinct, characterized by a fibrous, proteinaceous connective tissue network that imparts thermal stability, thereby inhibiting rapid liquefaction at elevated temperatures. This structural integrity is hypothesized to confer potential pharmacological properties.

The primary fatty acid profile of Camelus dromedarius hump fat predominantly comprises palmitic acid (C16:0, 33.8 mol %), stearic acid (C18:0, 25.9 mol %), oleic acid (C18:1, 18.1 mol %), and myristic acid (C14:0, 6.3 mol %) (Haasmann 1998). Notably, the total saturated fatty acid content is 74.2%, surpassing the saturation levels found in conventional lipid sources. This elevated saturation contributes to the increased melting point of camel adipose tissue in comparison to porcine fat (Haasmann 1998).

Earlier investigations into the lipid composition of Camelus bactrianus revealed a higher degree of saturation relative to ovine and bovine tallow, predominantly attributable to an augmented stearic acid fraction and a concomitant reduction in oleic acid content (Gunstone and Russell, 1954). These findings underscore the evolutionary lipidomic adaptation of camelids to arid environments, optimizing fat storage efficiency and metabolic utilization under extreme climatic conditions.

### SUMMARY OF THE INVENTION

The present invention provides compositions, methods of manufacture, and therapeutic methods involving camelid-derived bioactive lipids. In one aspect, the invention provides a composition comprising palmitoylethanolamide (PEA) isolated from camelid adipose tissue and formulated with a pharmaceutically acceptable carrier. In another aspect, the invention provides methods for isolating fatty acid ethanolamides including PEA from camel adipose tissue using solvent extraction and chromatographic purification techniques. In another aspect, the invention provides therapeutic methods for treating pain, inflammation, burns, dermatological disorders, musculoskeletal disorders, and neuropathic conditions using camel-derived lipid compositions. In certain embodiments, the compositions further include additional endocannabinoid-related lipids such as oleoylethanolamide (OEA), arachidonoylethanolamine (AEA), and 2-arachidonoylglycerol (2-AG), which may act synergistically to enhance analgesic and anti-inflammatory effects.

### BRIEF DESCRIPTION OF THE DRAWINGS

The present invention is illustrated through the accompanying figures, which provide a detailed visualization of the disclosed methodology and experimental findings:

FIG. 1A is a flowchart illustrating the method of the present disclosure for isolation and purification of N-Palmitoylethanolamide (PEA) from Camelus species hump fat.

FIG. 1B is a chromatographic profile of an endocannabinoids group from Camelus species hump fat prepared according to the flowchart provided in FIG. 1A.

FIG. 2 illustrates detailed latency times and comparative analgesic efficacy of a composition of the present invention.

### DETAILED DESCRIPTION OF THE INVENTION

The present invention relates to an analgesic of endocannabinoid compounds, with a particular focus on palmitoylethanolamide (PEA) isolated from camel fat. The disclosed invention is camel fat-derived PEA as a novel, natural, and potent therapeutic agent for pain management, offering an alternative approach to existing analgesic therapies.

By leveraging advanced lipid isolation and purification techniques, the present invention provides a high-purity, bioactive formulation of camel-derived PEA with enhanced bioavailability, pharmacokinetic stability, and therapeutic efficacy. The disclosed methodologies facilitate the efficient utilization of camel hump fat as a sustainable source of bioactive lipids, offering a non-opioid alternative for analgesia and a potential replacement for synthetic pharmaceutical agents.

Furthermore, the invention introduces an innovative biotechnological approach to harness the anti-inflammatory, neuroprotective, and tissue-regenerative properties of camel-derived PEA, establishing its application in wound healing, musculoskeletal disorders, chronic pain syndromes, and dermatological protection. The extraction, purification, and formulation strategies as disclosed herein ensure maximum therapeutic potential, safety, and clinical viability, positioning camel hump fat as a novel, natural, and pharmacologically advantageous alternative to conventional analgesics and anti-inflammatory agents.

Camel hump fat contains a variety of fatty acid derivatives and lipid mediators that can be isolated and formulated for therapeutic use. Camel adipose tissue may be obtained from Camelus species including Camelus dromedarius or Camelus bactrianus, and the adipose tissue may originate from the dorsal hump or other lipid-rich tissues.

In one embodiment, camel adipose tissue is homogenized in a solvent mixture containing chloroform and methanol. The solvent system extracts lipophilic compounds including fatty acid ethanolamides. The homogenized mixture is centrifuged to separate aqueous and organic phases, and the organic phase contains lipid compounds including palmitoylethanolamide. Following extraction, the organic fraction may be evaporated under inert gas or vacuum. The residue may then be subjected to solid-phase extraction chromatography using C18 cartridges to isolate the fatty acid ethanolamide fraction. Further purification may be performed using chromatographic techniques such as high-performance liquid chromatography (HPLC). Analytical verification of compound identity may be conducted using liquid chromatography-tandem mass spectrometry (LC-MS/MS). The resulting purified lipid fraction may contain palmitoylethanolamide as well as related compounds including oleoylethanolamide, arachidonoylethanolamine, and 2-arachidonoylglycerol.

These compounds may be formulated into pharmaceutical or dermatological compositions. Formulations may include creams, gels, ointments, transdermal patches, injectable formulations, oral capsules, suspensions, emulsions, or nanoparticle-based delivery systems.

In certain embodiments, the compositions are applied topically to treat burn injuries, inflammatory skin conditions, musculoskeletal pain, arthritis, neuropathic pain, or post-surgical pain. The bioactive lipids may act through modulation of peroxisome proliferator-activated receptor alpha (PPAR-α), endocannabinoid signaling pathways, and inflammatory cytokine pathways.

1. Method for Isolation and Purification of Endocannabinoid Compounds from Camel Hump Fat

A key aspect of this invention is the isolation of PEA from camel hump fat through a dual-phase extraction strategy, ensuring both high purity and optimal yield. The method comprises:
Liquid-Liquid Extraction (LLE)—Facilitates the initial separation of lipid-soluble components from the crude fat matrix.
Solid-Phase Extraction (SPE)—Further refines the PEA fraction, eliminating non-essential lipid constituents and maximizing bioavailability.

### 2. Analytical Quantification and Validation of PEA

To ensure the structural integrity and quantification of the extracted PEA, the present invention utilizes Liquid Chromatography-Tandem Mass Spectrometry (LC-MS/MS). This analytical approach allows for precise molecular characterization, verifying the purity, stability, and bioactivity of the compound.

### 3. Therapeutic Applications of Camel Fat-Derived PEA

The present invention discloses a topical analgesic formulation containing camel-derived PEA, specifically designed for pain relief. Given its anti-inflammatory and neuroprotective effects, camel-derived PEA serves as a viable alternative to opioid-based analgesics, minimizing dependency risks and adverse side effects associated with conventional pain management therapies.

### 4. Compositions for Treating Burns, Arthritis, and Post-Surgical Conditions

In one aspect, the present invention provides a therapeutic composition comprising fat derived from the hump of a camel, formulated for the treatment of:
Burn injuries, promoting tissue regeneration and reducing inflammation.
Arthritis and musculoskeletal disorders, alleviating joint inflammation and pain.
Post-surgical recovery, accelerating wound healing and mitigating inflammatory responses.

In one embodiment, the fat derived from the hump of a camel is enriched with N-Palmitoylethanolamide (PEA) to enhance its therapeutic efficacy.

### 5. Methods of Preparation for Camel Hump Fat-Derived Compositions

The present invention also provides a controlled thermal processing method for optimizing the extraction of bioactive lipid fractions. The method involves:
a) Heating camel hump fat under controlled conditions to increase lipid bioavailability and facilitate impurity separation:
   60° C. for 45 minutes, or
   100° C. for 20 minutes.
b) Post-heating purification, ensuring optimal retention of bioactive compounds while removing unwanted components.

### 6. Methods of Administration for Therapeutic Applications

The present invention provides a method for treating subjects suffering from burns, arthritis, and physical pain through the administration of camel hump fat-derived formulations.

In one embodiment, the method includes topical application, oral administration, or injectable delivery for the treatment of pain such as the following examples.

### Types of Pain Treated by the Present Invention

### Head and Facial Pain

Sinus Pain—Pressure in the forehead, cheeks, and around the eyes due to sinus inflammation.
Trigeminal Neuralgia—Severe, shock-like facial pain caused by trigeminal nerve irritation.
Temporomandibular Joint (TMJ) Pain—Dysfunction of the jaw joint causing pain in the jaw, temples, and ears.
Toothache—Pain from dental decay, infection, or nerve damage.

### Neck and Shoulder Pain

Cervical Spondylosis—Age-related cervical spine degeneration causing stiffness and pain.
Cervical Radiculopathy (Pinched Nerve)—Nerve compression in the neck radiating pain to the arms.
Muscle Strain—Localized pain from overuse or injury.
Whiplash—Trauma-induced pain from rapid back-and-forth neck movement.
Frozen Shoulder (Adhesive Capsulitis)—Joint stiffness and pain due to inflammation.

### Upper Limb Pain

Rotator Cuff Injury—Tear or inflammation of shoulder tendons.
Tennis Elbow (Lateral Epicondylitis)—Outer elbow pain from repetitive strain.
Golfer's Elbow (Medial Epicondylitis)—Inner elbow pain from overuse.
Carpal Tunnel Syndrome—Wrist nerve compression leading to pain, tingling, and numbness.
Arthritis (Osteoarthritis, Rheumatoid Arthritis)—Inflammatory joint pain affecting shoulders, elbows, wrists, and fingers.

### Spinal and Lower Limb Pain

Herniated Disc—Protruding spinal disc causing nerve compression and pain.
Sciatica—Pain radiating along the sciatic nerve from the lower back to the legs.
Spondylolisthesis—Vertebral slippage causing lower back pain.
Spinal Stenosis—Spinal canal narrowing leading to nerve compression.
Lumbago (General Lower Back Pain)—Muscular or structural lower back pain.

### Lower Limb Pain

Hip Pain (Osteoarthritis, Bursitis, Labral Tear)—Chronic hip joint or soft tissue pain.
Thigh Pain (Meralgia Paresthetica, Muscle Strains)—Nerve compression or muscle strain-related pain.
Knee Pain (ACL Tear, Meniscus Tear, Arthritis, Patellar Tendinitis)—Injury or inflammation causing knee joint pain.
Shin Splints (Medial Tibial Stress Syndrome)—Pain along the shin bone from overuse.
Plantar Fasciitis—Heel pain due to inflammation of the plantar fascia.
Peripheral Neuropathy—Nerve damage causing burning, tingling, or shooting pain in the lower extremities.

### Chronic and Systemic Pain Conditions

Fibromyalgia—Widespread musculoskeletal pain, fatigue, and sleep disturbances.
Chronic Fatigue Syndrome (CFS/ME)—Persistent pain with fatigue and cognitive dysfunction.
Complex Regional Pain Syndrome (CRPS)—Severe nerve pain following trauma or surgery.
Myofascial Pain Syndrome—Deep muscle pain due to trigger points.
Autoimmune Pain (Lupus, Rheumatoid Arthritis, Multiple Sclerosis)—Pain from systemic inflammation and immune dysfunction.
Cancer-related Pain—Pain from tumor growth or cancer treatment-related effects.
Anesthetic and pain reliever in surgical procedures and other medical treatment.

### 7. Photoprotective and Sunscreen Applications

In an additional aspect, the present invention provides a method for formulating a photoprotective composition (sunscreen) using camel hump fat-derived lipids. The formulation is prepared by:
a) Thermally processing camel hump fat under controlled conditions:
   60° C. for 45 minutes, or
   100° C. for 20 minutes.
b) Purification of the lipid fraction, ensuring optimal retention of photoprotective and anti-inflammatory properties.

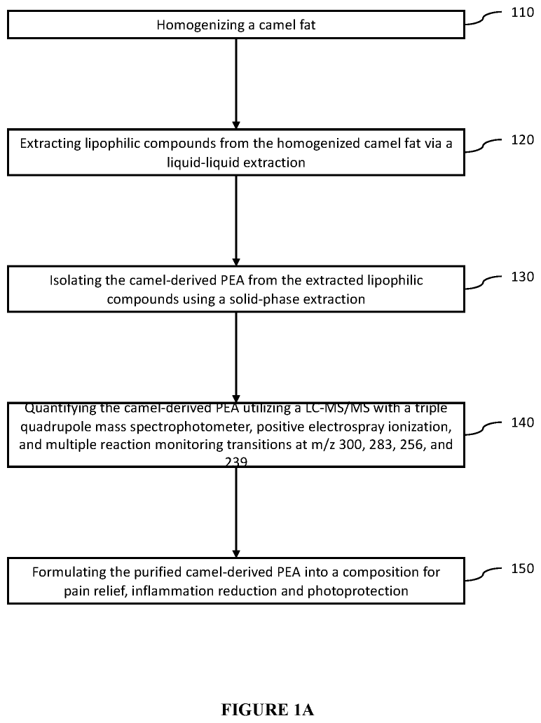

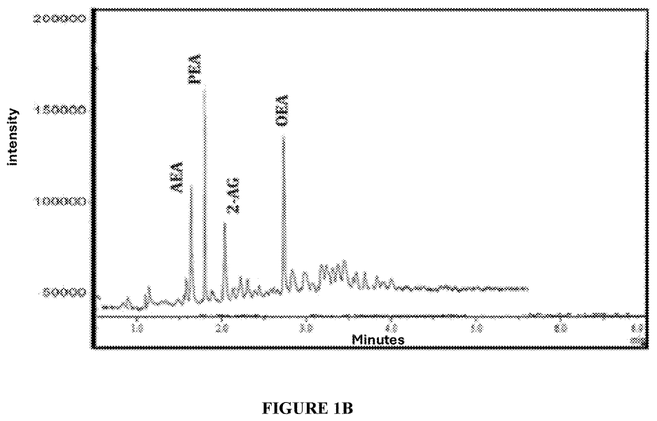

Referring to FIG. 1A, a process flowchart is provided illustrating a method for isolation and purification of N-Palmitoylethanolamide (PEA) from Camelus species hump fat, highlighting the sequential steps involved in liquid-liquid extraction (LLE) and solid-phase extraction (SPE).

Referring to FIG. 1B, a chromatographic profile is provided of endocannabinoids group in camel fat, prepared as shown in FIG. 1A: LC-MS/MS analysis of AEA, PEA, 2-AG, and OEA demonstrating the retention time, purity profile, and spectral characteristics of the extracted endocannabinoids, and particularly the PEA, confirming its structural integrity and chemical composition

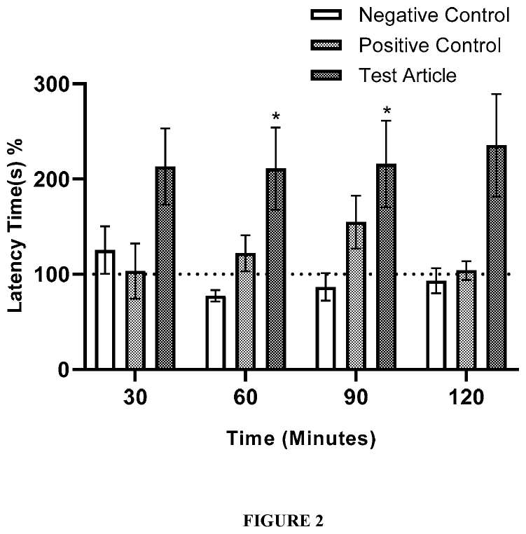

Referring to FIG. 2, detailed latency times and comparative analgesic efficacy is shown for all experimental groups, including the negative and positive control. In FIG. 2, the percentage change in latency time using Eddy's hot plate model after topical application of vehicle (Negative Control, NC), Emla® cream (Positive Control, PC) and the Test Article (TA) is shown. The dotted line represents the baseline reading of non-treated mice. *: p<0.05 in comparison to the NC group. Data is presented as mean±SEM (n=8).

### Endocannabinoid Extraction and LC-MS/MS Quantitation

### Extraction Procedure

A dual-phase extraction and purification methodology was employed to isolate PEA from camel hump fat, utilizing liquid-liquid extraction (LLE) followed by solid-phase extraction (SPE-C18), with subsequent LC-MS/MS quantitation.
1. Homogenization—Camel fat tissue (100 mg) was homogenized in 5 mL chloroform/methanol (2:1, v/v).
2. Vortexing—The homogenate was vortexed for 4 minutes.
3. Centrifugation—The mixture was centrifuged at 4,000 RPM for 10 minutes, separating organic and aqueous phases.
4. Solvent Evaporation—The organic layer was collected and evaporated to dryness under nitrogen.
5. Reconstitution—The dried residue was dissolved in 2 mL methanol for purification.

### Solid-Phase Extraction (SPE)

1. Sample Loading—The reconstituted extract (2 mL) was loaded into an SPE-C18 cartridge.
2. Washing Steps—The column was sequentially washed with water (1 mL) and 5% methanol (1 mL).
3. Elution—The purified PEA fraction was selectively eluted using 1 mL methanol.
4. Final Preparation—The eluate was evaporated under vacuum/nitrogen and reconstituted in 200 μL mobile phase for LC-MS/MS analysis.

### LC-MS/MS Analysis

### Chromatographic and Mass Spectrometric Parameters

Column: Reverse-phase C18 (ACE, 4.5 mm×0.45 mm×15 cm)
Mobile Phase:
   A: Water+0.1% formic acid
   B: Acetonitrile+0.1% formic acid
Flow Rate: 0.3-0.5 mL/min
Retention Time for PEA: 4-8 minutes
Ionization Mode: Positive Electrospray Ionization (ESI+)
Precursor Ion (m/z 300)→Fragment Ions (m/z 283, 256, 239)
Collision Energy: 20-35 eV

Table 1 presents a comprehensive chromatographic profile of compound groups in camel fat, analyzed via LC-MS/MS. This analysis elucidates the structural integrity and chemical composition of camel oil, providing a detailed characterization of its molecular constituents.

TABLE 1

| Elements | % | mg/100 gm | mg/L | ng/L | ng/gm | ppm |
|---|---|---|---|---|---|---|
| Calcium (Ca) | 0.082 | 82 | 820 | 8.2 × 10⁸ | 820,000 | |
| Potassium (K) | 0.074 | 74 | 740 | 7.4 × 10⁸ | 740,000 | |
| Magnesium (Mg) | 0.018 | 18 | 180 | 1.8 × 10⁸ | 180,000 | |
| Sodium (Na) | 0.0135 | 13.5 | 135 | 1.35 × 10⁸ | 135,000 | |
| Phosphorus(P) | 0.0069 | 6.93 | 69.3 | 6.93 × 10⁷ | 69,300 | |
| Selenium (Se) | 0.0025 | 2.51 | 25.1 | 2.51 × 10⁷ | 25,100 | |
| Zinc (Zn) | 0.00106 | 1.06 | 10.6 | 1.06 × 10⁷ | 10,600 | |
| Iron (Fe) | 0.00053 | 0.53 | 5.3 | 5.3 × 10⁶ | 5,300 | |
| Manganese (Mn) | 0.000017 | 0.017 | 0.17 | 170,000 | 170 | |
| Copper (Cu) | 0.000013 | 0.013 | 0.13 | 130,000 | 130 | 0.13 |
| Cadmium (Cd) | 0.0000091 | 0.0091 | 0.091 | 91000 | 91 | 0.091 |
| Chromium (Cr) | 0.0000069 | 0.0069 | 0.069 | 69000 | 69 | 0.069 |
| Lead (pb) | 0.0000025 | 0.0025 | 0.025 | 25000 | 25 | 0.025 |

| Vitamins | % | mg/100 gm | mg/L | ng/L | ng/gm |
|---|---|---|---|---|---|
| A | 14.1 | 14,100 | 141,000 | 1.4 × 10¹¹ | 1.41 × 10⁸ |
| Beta carotene | 1.4 | 1,400 | 14,000 | 1.4 × 10¹⁰ | 1.4 × 10⁷ |
| “Converted to A” | | | | | |
| B₁ | 19.2 | 19,200 | 192,000 | 1.92 × 10¹¹ | 1.92 × 10⁸ |
| Niacin “B₃” | 4.3 | 4,300 | 43,000 | 4.3 × 10¹⁰ | 4.3 × 10⁷ |
| Pantothenic acid “B₅” | 0.9 | 900 | 9,000 | 9 × 10⁹ | 9 × 10⁶ |
| Biotin “B₇” | 1.2 | 1,200 | 12,000 | 1.2 × 10¹⁰ | 1.2 × 10⁷ |
| B₁₂ | 2.1 | 2,100 | 21,000 | 2.1 × 10¹⁰ | 2.1 × 10⁷ |
| C | 29 | 29,000 | 290,000 | 2.9 × 10¹¹ | 2.9 × 10⁸ |
| D | 13.4 | | 0.0134 | | |
| E | 25 | 25,000 | 250,000 | 2.5 × 10¹¹ | 2.5 × 10⁸ |
| K | 3.1 | 3,100 | 31,000 | 3.1 × 10¹⁰ | 3.1 × 10⁷ |

| Endocannabinoids | % | mg/100 gm | mg/L | ng/L | ng/gm | mg/gm |
|---|---|---|---|---|---|---|
| N-Palmitoylethanolamine (PEA) | 39 | 39,000 | 390,000 | 3.9 × 10¹¹ | 3.9 × 10⁸ | 390 |
| Oleoylethanolamide (OEA) | 28 | 28,000 | 280,000 | 2.8 × 10¹¹ | 2.8 × 10⁸ | 280 |
| N-Arachidonoylethanolamine (AEA) | 21 | 21,000 | 210,000 | 2.1 × 10¹¹ | 2.1 × 10⁸ | 210 |
| 2-Arachidonoylglycerol (2-AG) | 12 | 12,000 | 120,000 | 1.2 × 10¹¹ | 1.2 × 10⁸ | 120 |

| Saturated fatty acid | % | mg/100 gm | mg/L | ng/L | ng/gm |
|---|---|---|---|---|---|
| Butyric Acid (C4:0) | 8 | 8,000 | 80,000 | 8 × 10¹⁰ | 8 × 10⁷ |
| Hexanoic acid (Caproic acid) | 3.1 | 3,100 | 31,000 | 3.1 × 10¹⁰ | 3.1 × 10⁷ |
| (C6:0) | | | | | |
| Caprylic acid (C8:0) | 5 | 5,000 | 50,000 | 5 × 10¹⁰ | 5 × 10⁷ |
| Capric acid (C10:0) | 4.5 | 4,500 | 45,000 | 4.5 × 10¹⁰ | 4.5 × 10⁷ |
| Undecanoic acid (C11:0) | 6 | 6,000 | 60,000 | 6 × 10¹⁰ | 6 × 10⁷ |
| Lauric acid (12:0) | 6.6 | 6,600 | 66,000 | 6.6 × 10¹⁰ | 6.6 × 10⁷ |
| Tridecanoic acid (C13:0) | 5 | 5,000 | 50,000 | 5 × 10¹⁰ | 5 × 10⁷ |
| Myristic acid (C14:0) | 8 | 8,000 | 80,000 | 8 × 10¹⁰ | 8 × 10⁷ |
| Pentadecenoic acid (C15:1) | 4.1 | 4,100 | 41,000 | 4.1 × 10¹⁰ | 4.1 × 10⁷ |
| Pentadecylic acid (C15:0) | 2.2 | 2,200 | 22,000 | 2.2 × 10¹⁰ | 2.2 × 10⁷ |
| Margaroleic acid (C17:1) | 2.4 | 2,400 | 24,000 | 2.4 × 10¹⁰ | 2.4 × 10⁷ |
| Margaric acid (C17:0) | 3.6 | 3,600 | 36,000 | 3.6 × 10¹⁰ | 3.6 × 10⁷ |
| Stearic acid (C18:0) | 9.6 | 9,600 | 96,000 | 9.6 × 10¹⁰ | 9.6 × 10⁷ |
| Nonadecylic acid (C19:0) | 3.3 | 3,300 | 33,000 | 3.3 × 10¹⁰ | 3.3 × 10⁷ |
| Arachidic acid (C20:0) | 6 | 6,000 | 60,000 | 6 × 10¹⁰ | 6 × 10⁷ |
| Heneicosanoic acid (C21:0) | 3.3 | 3,300 | 33,000 | 3.3 × 10¹⁰ | 3.3 × 10⁷ |
| Docosanoic acid (C22:0 | 4.1 | 4,100 | 41,000 | 4.1 × 10¹⁰ | 4.1 × 10⁷ |
| Lignoceric acid (C24:0) | 3.5 | 3,500 | 35,000 | 3.5 × 10¹⁰ | 3.5 × 10⁷ |
| Palmatic acid (C16:0) | 12.4 | 12,400 | 124,000 | 1.24 × 10¹¹ | 1.24 × 10⁸ |
| Unsaturated fatty acid | % | mg/100 gm | mg/L | ng/L | ng/gm |
| Myristoleic acid (C14:1) | 19 | 19,000 | 190,000 | 1.9 × 10¹¹ | 1.9 × 10⁸ |
| Omega-6 | 0.8 | 800 | 8,000 | 8 × 10⁹ | 8 × 10⁶ |
| Palmitoleic acid (C16:1) | 6 | 6,000 | 60,000 | 6 × 10¹⁰ | 6 × 10⁷ |
| Linolenic acid (C18:3n-3) | 5 | 5,000 | 50,000 | 5 × 10¹⁰ | 5 × 10⁷ |
| Linoleic acid (C18:2n-6) | 3 | 3,000 | 30,000 | 3 × 10¹⁰ | 3 × 10⁷ |
| Oleic acid (C18:1) | 22 | 22,000 | 220,000 | 2.2 × 10¹¹ | 2.2 × 10⁸ |
| Eicosapentaenoic acid | 4.1 | 4,100 | 41,000 | 4.1 × 10¹⁰ | 4.1 × 10⁷ |
| (C20:5n-3) | | | | | |
| Omega- 3 | 1.2 | 1,200 | 12,000 | 1.2 × 10¹⁰ | 1.2 × 10⁷ |
| Arachidonic acid | 3.2 | 3,200 | 32,000 | 3.2 × 10¹⁰ | 3.2 × 10⁷ |
| (C20:4n-6), | | | | | |
| Eicosatrienoic acid (20:3n-3) | 6.2 | 6,200 | 62,000 | 6.2 × 10¹⁰ | 6.2 × 10⁷ |
| Eicosadienoic acid | 5 | 5,000 | 50,000 | 5 × 10¹⁰ | 5 × 10⁷ |
| (C20:2n-6) | | | | | |
| Gondoic acid (C20:1) | 9 | 9,000 | 90,000 | 9 × 10¹⁰ | 9 × 10⁷ |
| Cervonic acid (C22:6n-3) | 2.2 | 2,200 | 22,000 | 2.2 × 10¹⁰ | 2.2 × 10⁷ |
| Erucic acid (C22:1) | 4 | 4,000 | 40,000 | 4 × 10¹⁰ | 4 × 10⁷ |
| Nervonic acid (C24:1) | 8 | 8,000 | 80,000 | 8 × 10¹⁰ | 8 × 10⁷ |
| omega 9 | 1 | 1,000 | 10,000 | 1 × 10¹⁰ | 1 × 10⁷ |

| Triglyceride | % | mg/100 gm | mg/L | ng/L | ng/gm |
|---|---|---|---|---|---|
| Palmitin | 7.4 | 7,400 | 74,000 | 7.4 × 10¹⁰ | 7.4 × 10⁷ |
| Cholesterol | 1.2 | 1,200 | 12,000 | 1.2 × 10¹⁰ | 1.2 × 10⁷ |
| Tricaprin | 23 | 23,000 | 230,000 | 2.3 × 10¹¹ | 2.3 × 10⁸ |
| Trilaurin | 25 | 25,000 | 250,000 | 2.5 × 10¹¹ | 2.5 × 10⁸ |
| Trimyristin | 20 | 20,000 | 200,000 | 2 × 10¹¹ | 2 × 10⁸ |
| Stearin | 10 | 10,000 | 100,000 | 1 × 10¹¹ | 1 × 10⁸ |
| Tripalmitin | 13 | 13,000 | 130,000 | 1.3 × 10¹¹ | 1.3 × 10⁸ |

| Details lipid profiling | Concentration |
|---|---|
| Hex2cer | 0.72 * 10⁻⁴ ng/mg |
| (Dihexosylceramide) | |
| DG (Diglyceride) | 0.35 * 10⁻⁴ ng/mg |
| PC (PhosphatidylCholine) | 2.1 * 10⁻⁴ ng/mg |
| Hexcer (Hexosylceramide) | 0.6 * 10⁻⁴ ng/mg |
| CL (Cephalin) | 0.22 * 10⁻⁴ ng/mg |

| Collagen Test | Percent |
|---|---|
| Collagen | 6.27% |

| Amino acid 18 aa | %(g/100 g) |
|---|---|
| Alanine | 3.3 |
| Arginine | 5.1 |
| Aspartic acid | 7.2 |
| Cysteine | 1.5 |
| Glutamic acid | 21.1 |
| Glycine | 1.2 |
| Histidine | 2.9 |
| Isoleucine | 4.9 |
| Leucine | 9 |
| Lysine | 6.6 |
| Methionine | 2.6 |
| Phenylalanine | 3.7 |
| Proline | 13 |
| Serine | 3 |
| Threonine | 5.3 |
| Tyrosine | 3 |
| Tryptophan | 1.8 |
| Valine | 4.8 |

| Organic acid | %(g/100 g) |
|---|---|
| Lactic acid | 3.2 |
| Citric acid | 2.1 |
| Acetic acid | 1.8 |
| Butyric acid | 3.4 |
| Propionic acid | 4.2 |

Note:
International Accepted ppm for: Copper 1.3 ppm, Cadmium 15 ppm, Chromium 100 ppm Lead 10 ppm

From Table 5, several components in camel oil exhibit analgesic (pain-relieving) properties due to their roles in neurotransmission, inflammation modulation, and endogenous opioid system interactions. These include:

### 1. Elements:

Magnesium (Mg)—Plays a role in pain modulation and is used to alleviate migraines and neuropathic pain.
Zinc (Zn)—Supports immune function and has some pain-modulating effects.
Copper (Cu)—Has anti-inflammatory properties and may assist in pain relief.

### 2. Vitamins:

Vitamin B1 (Thiamine)—Supports nerve health and has been used to reduce neuropathic pain.
Vitamin B3 (Niacin/Nicotinamide)—Improves blood flow and has anti-inflammatory effects that may reduce pain.
Vitamin B12 (Cobalamin)—Essential for nerve function and has been shown to relieve neuropathic pain.
Vitamin C (Ascorbic Acid)—Has antioxidant and anti-inflammatory properties that help reduce pain, especially in post-surgical recovery.
Vitamin D—Plays a role in reducing chronic pain by modulating inflammation and nerve function.
Vitamin E—Acts as an antioxidant and reduces oxidative stress-related pain.
Vitamin K—Helps with anti-inflammatory processes that may aid in pain relief.

### 3. Endocannabinoids

N-Palmitoylethanolamine (PEA)
Oleoylethanolamide (OEA)
N-Arachidonoylethanolamine (AEA)
2-Arachidonoylglycerol (2-AG)

### 4. Saturated Fatty Acids (SFAs)

The potential analgesic (pain-relieving) properties, primarily due to their role in modulating inflammation, nerve function, and endogenous pain pathways. The most notable SFAs found in camel oil with analgesic properties include:
Lauric Acid (C12:0)—Exhibits anti-inflammatory and antimicrobial properties, potentially aiding in the reduction of pain associated with infections or inflammatory conditions.
Myristic Acid (C14:0)—Involved in cellular signaling and may have indirect effects on pain modulation through immune system interactions.
Stearic Acid (C18:0)—Known for its potential role in cell membrane integrity and inflammation modulation, which can influence nociceptive signaling.
Palmitic Acid (C16:0)—Demonstrates neuroprotective and anti-inflammatory effects, which may contribute to pain relief by modulating immune responses and reducing neuroinflammation.

While these SFAs contribute to anti-inflammatory and analgesic effects, their efficacy is often enhanced when acting synergistically with other lipid-based bioactive compounds, such as polyunsaturated fatty acids (PUFAs), ceramides, and phospholipids.

### 5. Unsaturated Fatty Acids (UFAs):

The analgesic properties, primarily through their anti-inflammatory effects, modulation of pain pathways, and influence on neurotransmitter function. The most notable unsaturated fatty acids with analgesic potential found in camel oil include:

### Omega-3 Polyunsaturated Fatty Acids (PUFAs)

Eicosapentaenoic Acid (EPA, C20:5n-3)—Reduces inflammation and pain by inhibiting pro-inflammatory cytokines and eicosanoids, particularly prostaglandins and leukotrienes.
Docosahexaenoic Acid (DHA, C22:6n-3) is another name “cervonic acid”—Modulates neuroinflammation and enhances endogenous pain relief mechanisms.
Alpha-Linolenic Acid (ALA, C18:3n-3)—Precursor to EPA and DHA, contributing to anti-inflammatory and analgesic effects.

### Omega-9 Monounsaturated Fatty Acids (MUFAs)

Palmitoleic Acid (C16:1)—Associated with anti-inflammatory effects, potentially reducing nociceptive pain.
Oleic Acid (C18:1)—Displays anti-inflammatory properties by regulating pro-inflammatory cytokines and oxidative stress, contributing to pain relief.

Among these, omega-3 PUFAs (EPA and DHA) are the most widely studied for their analgesic and anti-inflammatory properties, particularly in conditions like neuropathic pain, arthritis, and chronic inflammation. Their effects are often enhanced when combined with saturated fatty acids and bioactive lipids in natural sources such as camel oil.

### 6. Triglyceride:

Triglycerides themselves do not directly act as analgesics, but their fatty acid composition determines their impact on pain. Triglycerides rich in omega-3 PUFAs (DHA, EPA) or anti-inflammatory omega-6s (GLA, DGLA) can support analgesic effects, whereas those high in pro-inflammatory fatty acids (Arachidonic Acid) may exacerbate pain.

### 7. Details Lipid Profiling:

Among the listed compounds, Hex2Cer (Dihexosylceramide) and HexCer (Hexosylceramide) have been studied for potential roles in pain modulation, but HexCer (Hexosylceramide) is more commonly associated with analgesic properties. Certain glycosphingolipids, including hexosylceramides, are involved in cellular signaling that can influence pain perception.

However, PC (Phosphatidylcholine), DG (Diglyceride), and CL (Cephalin) are primarily involved in membrane structure, lipid metabolism, and signaling but are not known for direct analgesic properties.

### 8. Collagen:

Collagen has significant analgesic effects, especially for joint pain, osteoarthritis, tendon injuries, and muscle recovery. Its ability to reduce inflammation, repair connective tissue, and support joint function makes it a valuable natural option for pain management.

### 9. Amino Acids:

Several amino acids exhibit analgesic (pain-relieving) properties by acting on neurotransmitter systems, reducing inflammation, or modulating pain pathways were found in camel oil. Here are the most notable ones: Arginine, Cysteine, Glycine, Histidine, Tyrosine, Tryptophan. The amino acids with the most significant direct analgesic effects are arginine, glycine, and tryptophan, due to their roles in neurotransmitter modulation, anti-inflammatory actions, and pain signal regulation. Other amino acids, such as cysteine, histidine and tyrosine, contribute to pain relief by supporting antioxidant defense, neurotransmitter balance, and immune modulation.

### 10. Organic Acids:

Among the listed organic acids, butyric acid has the strongest analgesic effects, followed by propionic acid and lactic acid due to their anti-inflammatory and neuromodulatory properties. Citric acid and acetic acid have minimal direct analgesic effects but may influence metabolic pathways linked to inflammation.

### CONCLUSIONS

The present invention introduces a novel biotechnological approach for isolating, purifying, and formulating camel-derived palmitoylethanolamide (PEA) for pain relief, inflammation reduction, and photoprotection. The findings confirm PEA's efficacy as a non-opioid analgesic alternative, providing a safer pharmacological option for managing chronic pain, arthritis, and thermal injuries.

When human skin is treated with camel oil containing PEA, blood flow improves within 30 seconds, with the increased circulation lasting for more than six hours. This enhanced blood flow delivers essential nutrients from camel oil to the tissues, effectively reducing and alleviating burn-related pain due to the presence of PEA while promoting natural skin recovery and a healthy appearance.

Furthermore, PEA derived from camelid hump fat may serve as a novel drug delivery mechanism to tissues. Once a drug enters systemic circulation, its distribution across tissues varies due to factors such as blood perfusion, tissue binding (e.g., lipid content), regional pH, and cell membrane permeability. The rate at which a drug enters a tissue depends on blood flow, tissue mass, and partition characteristics between blood and tissue. Distribution equilibrium is achieved more rapidly in highly vascularized areas unless diffusion across cell membranes is the limiting factor. Following equilibrium, drug concentrations in tissues and extracellular fluids mirror plasma concentrations, while metabolism and excretion occur simultaneously, making the process dynamic and complex.

In most tissues, drug distribution is primarily determined by perfusion. In poorly perfused tissues, such as muscle and fat, distribution occurs more slowly, particularly if the tissue has a high affinity for the drug. Camelid oil and camel fat-derived palmitoylethanolamide (PEA), N-arachidonoylethanolamine (AEA), 2-arachidonoylglycerol (2-AG), and oleoylethanolamide (OEA) (FIG. 1B and Table 1) present a promising alternative to corticosteroids for pain relief by primarily reducing inflammation, a key contributor to pain in conditions such as arthritis, muscle injuries, and nerve irritation. By decreasing inflammation, they effectively alleviate pain. Chromatographic profiling of the endocannabinoid group in camel fat confirms the presence of N-arachidonoylethanolamine (AEA), PEA, 2-arachidonoylglycerol (2-AG), and oleoylethanolamide (OEA), demonstrating the structural integrity and chemical composition of these bioactive lipids within camel oil (FIG. 1B and Table 2).

TABLE 2

| | Molecular | Retention | |
|---|---|---|---|
| Endocannabinoids | formula | time | mg/gm |
| N-Palmitoylethanolamine (PEA) | C₁₈H₃₇NO₂ | 39 | 390 |
| Oleoylethanolamide (OEA) | C₂₀H₃₉NO₂ | 28 | 280 |
| N-Arachidonoylethanolamine (AEA) | C₂₂H₃₇NO₂ | 21 | 210 |
| 2-Arachidonoylglycerol (2-AG) | C₂₃H₃₈O₄ | 12 | 120 |

The combination of AEA, PEA, 2-AG, and OEA in camel oil acts synergistically to relieve pain within minutes. AEA plays a significant role in modulating neuropathic, inflammatory, and chronic pain, making it a potential therapeutic target for pain management (Clapper et al. 2010; Donvito et al. 2018; Police et al. 2023). 2-AG possesses analgesic properties as an endocannabinoid, with research indicating its role in suppressing pain signaling, particularly in inflammatory and neuropathic pain (Martinez-Rivera et al. 2024; Singh et al. 2024).

Additionally, PEA is well-documented for its neuroprotective and analgesic properties, influencing neurotransmitter levels, emotional regulation, and pain perception. One of PEA's key mechanisms involves the activation of peroxisome proliferator-activated receptor-alpha (PPAR-α), which regulates monoamine neurotransmitters such as serotonin (5-HT). This modulation significantly alters serotonin levels in the basal forebrain, suggesting a role in emotional regulation and stress adaptation (Murillo-Rodriguez et al. 2021). Research further suggests that PEA may alleviate negative emotional states induced by morphine withdrawal by mitigating reductions in monoamine neurotransmitter levels in critical brain regions, including the hippocampus and prefrontal cortex (Wei et al. 2024). Despite its therapeutic potential, the isolation and pharmacological evaluation of camel fat-derived PEA remain unexplored.

OEA also contributes to pain relief through its interactions with PPAR-α receptors and involvement in endocannabinoid system modulation. It exhibits analgesic properties, reducing inflammatory and neuropathic pain by decreasing pain signaling and inflammation (Suardiaz et al. 2007; Payahoo et al. 2018; Pérez-Martin et al. 2023). Additionally, OEA exerts an anorexic effect, enhances fatty acid absorption in the small intestine, and stimulates lipolysis through PPAR-α activation (Hussain et al. 2017). These findings highlight the potential of camel oil or camel fat-derived PEA and OEA as therapeutic candidates for managing both pain and metabolic functions.

Given the collective analgesic and anti-inflammatory properties of AEA, PEA, 2-AG, and OEA, camelid oil offers a promising natural alternative for pain management. Further research into the pharmacological actions of camel fat-derived endocannabinoids may provide deeper insights into their therapeutic potential across various medical applications.

In general, the synergistic analgesic effects of camel-derived lipids provide a viable, non-opioid alternative for pain management, particularly in chronic and inflammatory conditions.

The invention has been described herein, but it is evident that variations may be made without departing from its spirit and scope. Any modifications that would be apparent to a person skilled in the art are intended to fall within the scope of the following claims.

### REFERENCES

Agour, A.; Mssillou, I.; El Barnossi, A.; Chebaibi; et al. 2023. Extracts of Brocchia cinerea (delile) vis exhibit in vivo wound healing, anti-inflammatory and analgesic activities, and other in vitro therapeutic effects. Life, 13:776. https://doi.org/10.3390/life13030776.
Amerikanou, C., and Papada, E. 2023. Plant-derived natural products and their biomedical properties: Recent advances and future directions. Life 13:2105. https://doi.org/10.3390/life13102105.
Capó X, Martorell M, Tur J A, Sureda A, Pons A. 2021. 5-Dodecanolide, a Compound Isolated from Pig Lard, Presents Powerful Anti-Inflammatory Properties. Molecules. 26(23):7363. doi: 10.3390/molecules26237363. PMID: 34885945; PMCID: PMC8658921.
Clapper J R, Moreno-Sanz G, Russo R, et al. 2010. Anandamide suppresses pain initiation through a peripheral endocannabinoid mechanism. Nat Neurosci. (10):1265-70. doi: 10.1038/nn.2632. Epub 2010 Sep. 19. PMID: 20852626; PMCID: PMC3260554.
Diochot S 2021. Pain-related toxins in scorpion and spider venoms: a face to face with ion channels. J Venom Anim Toxins Incl Trop Dis. 27:e20210026. doi: 10.1590/1678-9199-JVATITD-2021-0026. PMID: 34925480; PMCID: PMC8667759.
Gunstone F D and Russell W C (1954) Animal fats: 4. The component acids of crocodile fat. Biochem J. 57(3): 462-465.
Donvito, G., Nass, S., Wilkerson, J. et al. 2018. The Endogenous Cannabinoid System: A Budding Source of Targets for Treating Inflammatory and Neuropathic Pain. Neuropsychopharmacol. 43, 52-79https://doi.org/10.1038/npp.2017.204
Haasmann S O (1998). Analytical characterization of camel meat and milk fat. A thesis submitted for the degree of Doctor of Philosophy. Department of Chemistry, Brunel University.
Hussain Z, Uyama T, Tsuboi K and Ueda N. 2017. Mammalian enzymes responsible for the biosynthesis of N-acylethanolamines. Biochimica et Biophysica Acta (BBA)—Molecular and Cell Biology of Lipids1862 (12):1546-1561
Lisa, S. R., Islam, M. K., and Qais, N. 2020. Plants and plant constituents with analgesic and anti-inflammatory activities: A Systematic Review. Dhaka University Journal of Pharmaceutical Sciences, 19(2):207-224. https://doi.org/10.3329/dujps.v19i2.50638.
Locci A and Pinna G. 2019. Stimulation of peroxisome proliferator-activated receptor-α by n-palmitoylethanolamine engages allopregnanolone biosynthesis to modulate emotional behavior, Biol. Psychiatry 85:1036-1045, https://doi.org/10.1016/j.biopsych.2019.02.006.
Martinez-Rivera A, Fetcho R N, Birmingham L, Xu J, et al. 2024. Elevating levels of the endocannabinoid 2-arachidonoylglycerol blunts opioid reward but not analgesia. Sci Adv. 10(48):eadq4779. doi: 10.1126/sciadv.adq4779. Epub 2024 Nov. 29. PMID: 39612328; PMCID: PMC11606496.
Murillo-Rodriguez E, Arankowsky-Sandoval G, Budde H, Imperatori C, Machado S, Yamamoto T, Yadollahpour A, Torterolo P. 2021. In vivo brain levels of acetylcholine and 5-hydroxytryptamine after oleoylethanolamide or palmitoylethanolamide administrations are mediated by PPARα engagement, Eur. J. Neurosci. 54:5932-5950, https://doi.org/10.1111/ejn.15409.
Pérez-Martin, E.; Pérez-Revuelta, L.; Barahona-López, C.; Pérez-Boyero, D.; Alonso, J. R.; Diaz, D.; Weruaga, E. 2023. Oleoylethanolamide treatment modulates both neuroinflammation and microgliosis, and prevents massive leukocyte infiltration to the cerebellum in a mouse model of neuronal degeneration. Int. J. Mol. Sci. 24, 9691. https://doi.org/10.3390/ijms24119691.
Payahoo L, Khajebishak Y, Asghari Jafarabadi M, Ostadrahimi A. 2018. Oleoylethanolamide supplementation reduces inflammation and oxidative stress in obese people: A clinical trial. Adv Pharm Bull. 8(3):479-487. doi: 10.15171/apb.2018.056. Epub 2018 Aug. 29. PMID: 30276145; PMCID: PMC6156479.
Police A, Shankar V K, Pandey P, Rangappa S, Doerksen R J, Narasimha Murthy S. 2023. Novel topical anandamide formulation for alleviating peripheral neuropathic pain. Int J Pharm. 25; 641:123085. doi: 10.1016/j.ijpharm.2023.123085. Epub 2023 May 26. PMID: 37245739.
Ramiro I B L, Bjorn-Yoshimoto W E, Imperial J S. Et al. 2022. Somatostatin venom analogs evolved by fish-hunting cone snails: From prey capture behavior to identifying drug leads. Sci Adv. 25; 8(12):eabk1410. doi: 10.1126/sciadv.abk1410. Epub 2022 Mar. 23. PMID: 35319982; PMCID: PMC8942377.
Singh, S., Ellioff, K. J., Bruchas, M. R., Land, B. B., & Stella, N. 2024. Analgesic properties of next generation modulators of endocannabinoid signaling: Leveraging modern tools for the development of novel therapeutics. The Journal of pharmacology and experimental therapeutics. 391(2):162-173.
Suardiaz M, Estivill-Torrús G, Goicoechea C, Bilbao A, Rodriguez de Fonseca F. 2007. Analgesic properties of oleoyletbanolamide (OEA) in visceral and inflammatory pain. Pain 133(1):99-110. DOI: 10.1016/j.pain.2007.03.008
Wang C., Chen M, Lu, X., Yang S., et al. 2023. Isolation and characterization of poeciguamerin, a peptide with dual analgesic and anti-thrombotic activity from the Poecilobdella manillensis leech. International Journal of Molecular Sciences. 24 (13):11097. https://doi.org/10.3390/ijms241311097
Wei Y B, Wang Y B, Sun J Y, Wang S, Nan J, Yu H L, Lan Y. 2024. N-palmitoylethanolamide attenuates negative emotions induced by morphine withdrawal in mice. Neurosci Lett. 15; 841:137944. doi: 10.1016/j.neulet.2024.137944. PMID: 39154843.
Zainal Z, Longman A J, Hurst S, et al. 2009. Relative efficacies of omega-3 polyunsaturated fatty acids in reducing expression of key proteins in a model system for studying osteoarthritis. Osteoarthritis Cartilage. 17:896-905. doi: 10.1016/j.joca.2008.12.009

1. A therapeutic composition comprising a camelid-derived lipid extract comprising palmitoylethanolamide (PEA) and a pharmaceutically acceptable carrier.

2. The composition of claim 1 wherein the lipid extract is derived from camel hump adipose tissue.

3. The composition of claim 1 wherein the composition further comprises at least one additional fatty acid ethanolamide.

4. The composition of claim 3 wherein the fatty acid ethanolamide is selected from oleoylethanolamide, arachidonoylethanolamine, and 2-arachidonoylglycerol.

5. The composition of claim 1 formulated as a topical pharmaceutical formulation.

6. The composition of claim 1 formulated as a cream, gel, ointment, spray, nanoparticle-based delivery system or transdermal patch.

7. The composition of claim 1 wherein the lipid extract retains a mixture of camel-derived fatty acids and lipid mediators.

8. A method of producing palmitoylethanolamide derived from camelid adipose tissue comprising:
homogenizing camel adipose tissue in a solvent system;
extracting lipophilic compounds;
purifying the extract using chromatographic separation; and
recovering palmitoylethanolamide.

9. The method of claim 8 wherein the solvent system comprises chloroform and methanol.

10. The method of claim 8 wherein purification comprises solid phase extraction.

11. The method of claim 8 further comprising LC-MS/MS analysis of the purified compound.

12. A method of treating pain in a subject comprising administering a therapeutically effective amount of a composition comprising camel-derived palmitoylethanolamide.

13. The method of claim 12 wherein the pain comprises inflammatory pain.

14. The method of claim 12 wherein the pain comprises neuropathic pain.

15. The method of claim 12 wherein the composition is administered topically.

16. The method of claim 12, wherein administering the composition comprises a nanoparticle-based delivery system.

17. The method of claim 12, wherein administering the composition comprises sustained-release topical gels.

18. The method of claim 12, wherein the therapeutically effective composition further comprises camelid-derived synergistic endocannibinoid mixtures.

19. The method of claim 12, wherein the composition comprises liposomal formulations.
